(19) United States
(12) Patent Application Publication
(10) Pub. No.: US 2008/0281368 A1
(43) Pub. Date: Nov. 13, 2008

(54) IMPLANTABLE DIGITAL DEVICE FOR TISSUE STIMULATION

(76) Inventors: Cherik Bulkes, Sussex, WI (US); Stephen Denker, Mequon, WI (US)

Correspondence Address: QUARLES & BRADY LLP, 411 E. WISCONSIN AVENUE, SUITE 2040, MILWAUKEE, WI, 53202-4497, US

(21) Appl. No.: 12/115,207

(22) Filed: May 5, 2008

Related U.S. Application Data
(60) Provisional application No. 60/916,851, filed on May 9, 2007.

(51) Int. Cl. A61N 1/39; A61N 1/368

(52) U.S. Cl. 607/4; 607/15; 607/11

(57) ABSTRACT

An implantable vagal stimulation device with high-energy efficiency and novel data sensing is provided for use in a wide variety of applications where neural stimulation is required, including human heart rate control. The stimulation device uses low-impedance circuitry and digital waveforms to minimize energy losses, thereby requiring a relatively small battery. Front-loaded, passive filtering is employed to reduce electromagnetic noise sensitivity, leaving a clear physiological signal without degradations. This physiological signal is processed by a derivative zero transition detector (DZD), which is immune to variations in input signal dynamic range unlike traditional methods. Information that the DZD receives can be then interpreted and used along with an algorithm to execute appropriate vagal nerve stimulation.

### CROSS-REFERENCE TO RELATED APPLICATIONS

This application claims benefit of U.S. Provisional Patent Application No. 60/916,851 filed May 9, 2007.

### STATEMENT REGARDING FEDERALLY SPONSORED RESEARCH OR DEVELOPMENT

Not Applicable

### BACKGROUND OF THE INVENTION

1. Field of Invention

The present invention relates to implantable medical devices which deliver energy to stimulate tissue in an animal, and more particularly to highly efficient stimulation devices that use digital stimulation output for use in a medical device that is implanted adjacent to tissue or organ.

2. Description of the Related Art

A remedy for people with slowed or disrupted natural heart activity is to implant a cardiac pacing device which is a small electronic apparatus that stimulates the heart to beat at regular rates.

Typically the pacing device is implanted in the patient's chest and has sensor electrodes that detect electrical impulses associated with in the heart contractions. These sensed impulses are analyzed to determine when abnormal cardiac activity occurs, in which event a pulse generator is triggered to produce electrical pulses. Wires carry these pulses to electrodes placed adjacent specific cardiac muscles, which when electrically stimulated contract the heart chambers. It is important that the stimulation electrodes be properly located to produce contraction of the heart chambers.

Modern cardiac pacing devices vary the stimulation to adapt the heart rate to the patient's level of activity, thereby mimicking the heart's natural activity. The pulse generator modifies that rate by tracking the activity of the sinus node of the heart or by responding to other sensor signals that indicate body motion or respiration rate.

US Published Patent Application No. 2008/0077184 describes an apparatus provided for artificially stimulating internal tissue of an animal by means of an intravascular medical device adapted for implantation into the animal's blood vasculature. The intravascular medical device comprises a power supply and a pair of stimulation electrodes for contacting the tissue. A control circuit governs operation of a stimulation signal generator connected to the pair of stimulation electrodes. The stimulation signal generator produces a series of electrical stimulation pulses and a voltage intensifier increases the voltage of each electrical stimulation pulse to produce an output pulse that is applied to the stimulation electrodes. One version of the medical device includes a mechanism that is connected to the stimulation electrodes for sensing effects from the electrical stimulation pulse and producing a feedback signal indicating such effects. Although this stimulation apparatus offered several advantages over other types of stimulators, it required energy efficient systems and highly robust signal sensing to be developed. Such energy efficient stimulation systems meet clinical needs of implanted stimulation devices with internal or external energy sources. An energy efficient stimulation system minimizes recharging requirements of external powered systems whereas it improves the battery lifetime of internally powered systems. It may also enable therapies that are not possible with current systems. Moreover, robust signal sensing will improve the signal detection and signal analysis tasks due to very low artifact content in the sensed signal.

Cardiac rhythm management systems include, among other items, pacemaker/defibrillators that combine the functions of pacemakers and defibrillators, drug delivery devices, and any other implantable or external systems or devices for diagnosing or treating cardiac arrhythmias.

One problem faced by cardiac rhythm management systems is the treatment of congestive heart failure (also referred to as “CHF”). Congestive heart failure, or heart failure, is a condition in which the heart can not pump enough blood to the body's other organs. This can result from various causes including narrowed arteries that supply blood to the heart muscle, the coronary artery disease; post heart attack, or myocardial infarction, with scar tissue that interferes with the heart muscle's normal work; high blood pressure; heart valve disease due to past rheumatic fever or other causes; primary disease of the heart muscle itself, called cardiomyopathy; heart defects present at birth—congenital heart defects; and infection of the heart valves and/or heart muscle itself—endocarditis and/or myocarditis.

The “failing” heart keeps working, but not as efficiently as it should. People with heart failure can not exert themselves because they become short of breath and tired. By way of example, suppose the muscle in the walls of the left side of the heart deteriorates. As a result, the left atrium and left ventricle become enlarged, and the heart muscle displays less contractility, often associated with unsynchronized contraction patterns. This decreases cardiac output of blood, and in turn, may result in an increased heart rate and less resting time between heart contractions. This condition may be treated by conventional dual-chamber pacemakers and a new class of biventricular (or multisite) pacemakers that are known as cardiac resynchronization therapy (CRT) devices. A conventional dual-chamber pacemaker typically paces and senses one atrial chamber and one ventricular chamber. A pacing pulse is timed to be delivered to the ventricular chamber at the end of a programmed atrio-ventricular delay, referred to as AV delay, which is initiated by a pace delivered to or an intrinsic depolarization detected from the atrial chamber. This mode of pacing is sometimes referred to as an atrial tracking mode. The heart can be paced with a lengthened AV delay to increase the resting time between heart contractions to increase the amount of blood that fills the ventricular chamber, thus increasing the cardiac output. Biventricular or other multisite CRT devices can pace and sense three or four chambers, usually including the right atrial chamber and both right and left ventricular chambers. By pacing both right and left ventricular chambers, the CRT device can restore a more synchronized contraction of the weakened heart muscle, thus increasing the heart's efficiency as a pump. When treating CHF with conventional CRT devices, it is critical to pace the both ventricular chambers continuously to provide resynchronizing pacing; otherwise, the patient will not receive the intended therapeutic benefit. Thus the intention for treating CHF patients with continuous pacing therapy is different from the intention for treating bradycardia patients with on-demand pacing therapy, which is active only when the heart's intrinsic (native) rhythm is abnormally slow.

Conventional pacemakers and CRT devices in current use rely on conventional on-demand pacing modes to deliver ventricular pacing therapy. These devices need to be adapted to provide a continuous pacing therapy required for treatment of CHF patients. One particular problem in these devices is that they prevent pacing when the heart rate rises above a maximum pacing limit. One such maximum pacing limit is a maximum tracking rate (MTR) limit. “MTR” and “MTR interval,” where an “MTR interval” refers to a time interval between two pacing pulses delivered at the MTR, are used interchangeably, depending on convenience of description, throughout this document. The MTR presents a problem particularly for CHF patients, who typically have elevated heart rates to maintain adequate cardiac output. When a pacemaker or CRT device operates in an atrial tracking mode, it senses the heart's intrinsic rhythm that originates in the right atrial chamber, that is, the intrinsic atrial rate. As long as the intrinsic atrial rate is below the MTR, the device will pace one or both ventricular chambers after an AV delay. If the intrinsic atrial rate rises above the MTR, the device will limit the time interval between adjacent ventricular pacing pulses to an interval corresponding to the MTR, that is, ventricular pacing rate will be limited to the MTR. In this case, the heart's intrinsic contraction rate is faster than the maximum pacing rate allowed by the pacing device so that after a few beats, the heart will begin to excite the ventricles intrinsically at the faster rate, which causes the device to inhibit the ventricular pacing therapy due to the on-demand nature of its pacing algorithm.

The MTR is programmable in most conventional devices so that the MTR can be set above the maximum intrinsic atrial rate associated with the patient's maximum exercise level, that is, above the physiological maximum atrial rate. However, many patients suffer from periods of pathologically fast atrial rhythms, called atrial tachyarrhythmia. Also some patients experience pacemaker-mediated tachycardia (PMT), which occurs when ventricular pacing triggers an abnormal retrograde impulse back into the atrial chamber that is sensed by the pacing device and triggers another ventricular pacing pulse, creating a continuous cycle of pacing-induced tachycardia. During these pathological and device-mediated abnormally elevated atrial rhythms, the MTR provides a protection against pacing the patient too fast, which can cause patient discomfort and adverse symptoms. Thus, to protect the patient against abnormally fast pacing, the MTR often is programmed to a low, safe rate that is actually below the physiological maximum heart rate. For many CHF patients with elevated heart rates, this means that they cannot receive the intended pacing therapy during high but physiologically normal heart rates, thus severely limiting the benefit of pacing therapy and the level of exercise they can attain. Therefore, there is a need for addressing this MTR-related problem in therapeutic devices for CHF patients as well as other patients for whom pacing should not be suspended during periods of fast but physiologically normal heart rates. Another problem encountered is that in some patients treated with CRT there is shortened conduction time between the atrium and the ventricle (shorten AV interval). In such cases, in order to permit CRT pacing the programmed AV interval has to be very short to permit resynchronization therapy. However, the same patients may benefit from a longer AV interval to permit increased cardiac filling. A means to therefore prolong the intrinsic AV interval to allow resynchronization therapy as well as increase filling is desirable.

### SUMMARY OF THE INVENTION

An apparatus is provided for artificially stimulating internal tissue of an animal by means of a medical device adapted for implantation in the animal. The medical device comprises a low impedance power supply and a plurality of stimulation leads and electrodes for contacting the tissue. A control circuit contained in the implanted enclosure, governs operation of a stimulation signal generator connected to the plurality of stimulation electrodes. The stimulation signal generator produces a series of electrical stimulation pulses for one or more given clinical purposes using specific predetermined waveforms. The stimulation circuit may include a voltage intensifier that increases the voltage of each electrical stimulation pulse to produce an output pulse that is applied to the stimulation electrodes. The stimulation lead with plurality of electrodes is designed to be a very low impedance structure to minimize power losses in the lead. The device may be used for vagal stimulation to slow down the ventricular rate so that therapy may be optimized for patients with more rapid rhythm which would otherwise inhibit CRT. Additionally, vagal stimulation may allow for appropriate ventricular filling in CHF patients.

The voltage intensifier can use any of several techniques to increase the stimulation pulse voltage from a standard low voltage implant battery, e.g. a three volt battery, contained within the implanted enclosure. Preferably, flying capacitor type voltage doubling, bipolar mode doubling, or a combination of both is used.

One version of the medical device includes a mechanism that is connected to plurality of stimulation electrodes for sensing effects from the electrical stimulation pulse and producing a feedback signal indicating such effects. The stimulation pulses are altered in response to the feedback signal, thereby controlling stimulation of the tissue.

The apparatus includes a low impedance power source that may be battery powered, or radio frequency based, or based on other forms of energy supply including but not limited to piezo electric devices, thermal energy sources, mechanical energy sources and chemical energy sources.

The medical device also can sense a physiological characteristic of the animal and send data related to the physiological characteristic via a wireless signal. The sensing device has no common ground reference and is, therefore, practically immune from noise sources that are inevitable in devices with a common ground. The output of the sensing circuit is analyzed by a derivative zero transition detector with a deadband which can further discriminate between noise from biological signals and the stimulation may be further controlled based on the detector output.

One version of the stimulation electrode assembly includes a dynamically programmable configuration to provide stimulation that can potentially mimic natural, biological stimulations.

The stimulation device further provides a digital output wherein the output voltage is chosen such that it is close to the desired output voltage. In such a device capture threshold is managed by modifying the duration of the digital output thereby minimizing losses even at the output stage, but also the structure of a compound multisegmented waveform, which may contain one or more waveform lobes, rather than a more traditional single or bipolar waveform.

### BRIEF DESCRIPTION OF DRAWINGS

FIG. 1 shows the anatomical references of the possible stimulation sites of the vagal nervous system in the fat pads of the epicardium;

FIG. 2 is a block schematic diagram of the electrical circuitry for a stimulation module according to the present invention;

FIG. 3 is a schematic diagram of a voltage intensifier in the intravascular medical device; and

FIG. 4 is a schematic diagram of a voltage inverter;

FIG. 5 illustrates a controller output signal applied to a voltage doubler to increase the amplitude of the output signal;

FIG. 6 depicts waveform diagrams related to bipolar stimulation signal generation;

FIG. 7 is a schematic diagram of high level modules in one embodiment of the stimulation system;

FIG. 8 is a schematic diagram of high level modules in another embodiment of the stimulation system;

FIG. 9 is an equivalent circuit diagram of the stimulation leads and tissue;

FIG. 10 illustrates a standard stimulation pulse produced by prior cardiac pacemakers;

FIG. 11 depicts one period of a composite stimulation pulse produced by the present stimulation system;

FIGS. 12A and B depict one period of an alternative composite stimulation pulse and a multi-lobe composite pulse;

FIG. 13 is a schematic diagram of a sensing amplifier with an internal reference and a high pass filter to reject DC and low frequency signals;

FIG. 14 shows the frequency response of the band pass filtering used in the stimulation system;

FIG. 15 is a schematic diagram of a sensing amplifier that has an internal reference and signal pre-filters;

FIG. 16 shows the details of the internal reference;

FIG. 17A shows exemplary waveforms at various nodes in a derivative zero transition detector schematically shown in FIG. 17B;

FIG. 18A shows a hysteresis waveform in another derivative zero transition detector schematically shown in FIG. 18B;

FIG. 19 shows an enhanced variant of the DZD depicted in FIG. 18B.

### DETAILED DESCRIPTION OF THE INVENTION

Although the present invention is being initially described in the context of cardiac pacing by implanting an intravascular radio frequency energy powered stimulator, the present apparatus comprising of a highly efficient stimulator with digital output, can be employed to stimulate one or more other areas of the human body as shown in subsequent descriptions and examples. Electrodes of the stimulator may be implanted in a vein or artery of the heart or it may be embedded in cardiac muscle or skeletal muscle. The stimulator may be configured to deliver treatment in the form of stimulation of the autonomous system, such as the cardiac vagal nerve for the purpose of heart rate control. In addition to cardiac applications, the stimulation apparatus can provide brain stimulation, for treatment of Parkinson's disease or obsessive/compulsive disorder for example. The electrical stimulation also may be applied to muscles, the spine, the gastro/intestinal tract, the pancreas, and the sacral nerve. The apparatus may also be used for GERD treatment, endotracheal stimulation, pelvic floor stimulation, treatment of obstructive airway disorder and apnea, molecular therapy delivery stimulation, chronic constipation treatment, and electrical stimulation for bone healing. The current invention can provide stimulation for two or more clinical purposes simultaneously as will be described later.

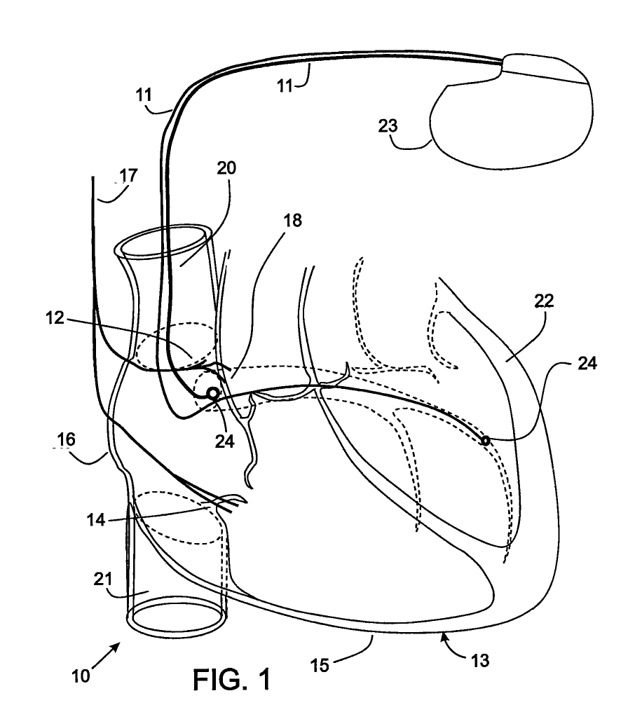

Reference FIG. 1, a medical device 10 is provided for artificially stimulating internal tissue, such as a heart 13 of an animal by means of a stimulator 23 adapted for implantation in the animal. A plurality of stimulation leads 11 connect electrodes 24 to the stimulator 23 for sensing electrical signals in the hear and for applying electrical stimulation pulses to the heart tissue. The medical device 10 may stimulate the vagal nerve 14, 17 near the proximal coronary sinus (CS) 18 or from the inferior vena cava (IVC) 21 at the entry 12 into the right atrium 16, or from the superior vena cava (SVC) 20 at the entry 12 into the right atrium. During a treatment procedure, stimulation electrodes 24 are placed at locations near the vagal nerve 14, 17, such that one or more electrodes from a plurality of electrodes are programmably selected for optimal vagal stimulation. The stimulation waveforms are programmed with respect to shape, duration and duty cycle for maximizing energy conservation and minimizing stimulation sensation to patient. The atrial fibrillation sensing and stimulation further involves sensing right atrium (RA) 16 and right ventricle (RV) 15 or left ventricle (LV) 22 and detecting when RA rate is faster than RV or LV rate.

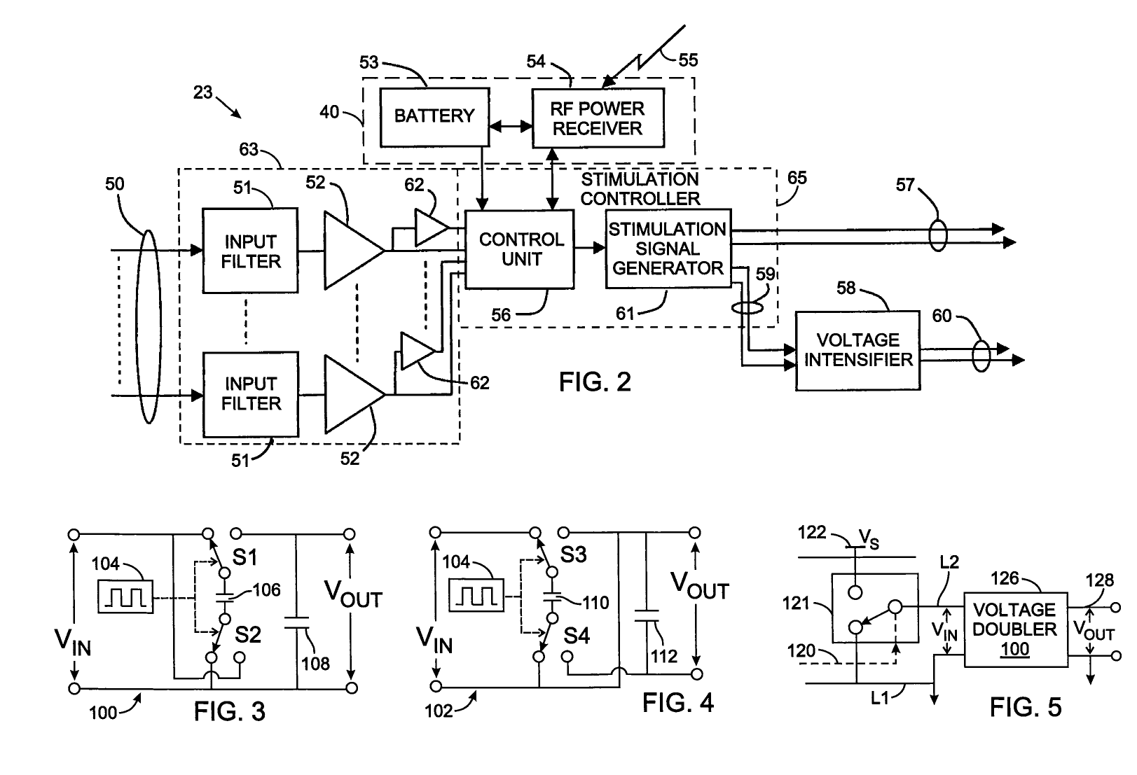

FIG. 2 schematically illustrates the circuitry in the stimulator 23. The stimulator 23 has a low impedance power supply 40 that comprises a battery 53 and a radio frequency (RF) transceiver 54 that derives electrical power from a received RF signal 55. Sensor electrodes 50 detect electrocardiogram signals and other physiological characteristics which are applied through input filters 51 to amplifiers 52 of a sensing unit 63. The outputs of the are fed directly to a control unit 56 and through differential zero detectors (DZD's) 62 to the control unit. The control unit preferably is a computerized device that executes a software program that analyzes the signals from the sensor electrodes 50 to determine when to stimulate the patient's vagal nerve or the heart itself.

When stimulation is desired, the control unit 56 issues a command to a stimulation signal generator 61, which are both part of a stimulation controller 65. Depending upon the desired treatment, the stimulation signal generator 61 applied an electrical pulse directly to a first set of electrodes 57 or drives a voltage intensifier 58 via connection 59 to apply a more intense stimulation pulse to a second set of electrodes 60. The voltage intensifier 58 may use any of several techniques to increase the stimulation pulse voltage from the standard low voltage implant battery 53, e.g. a three volt battery, contained within the implanted stimulator 23. Preferably, flying capacitor type voltage doubling, bipolar mode doubling, or a combination of both is used. The stimulation leads with plurality of electrodes are designed to be a very low impedance structure to minimize power losses in the leads.

By monitoring the physiological response in response to the stimulation from either pacing output electrodes 57 or nerve stimulation output electrodes 60, a feedback loop is formed which can be used to optimize the treatment or therapy.

For vagal nerve stimulation efficacy verification, the control unit 56 analyzes the sensed parameters to calculate the actual heart rate to determine whether the heart is pacing at the desired rate in response to the stimulation. If the heart is pacing at the desired rate, the control unit 56 can cease the stimulation. If pacing is needed, the pulse energy is adjusted in steps until pacing is no longer effective. The stimulation energy then is then set slightly above that threshold to minimize pacing energy and conserve battery power. Energy reduction can be accomplished at least in two ways: (1) preferably, the pulse duration is reduced to linearly decrease that amount of energy dissipated in the tissue, or (2) the voltage amplitude is reduced stepwise in situations where energy dissipation might vary non-linearly because the tissue/electrode interface impedance is unknown or unstable as is sometimes the case directly after implantation.

The stimulation is controlled by a functionally closed feedback loop. When stimulation commences, the sensed signal waveform can show a physiological response confirming effectiveness of that stimulation pulse. By stepwise increasing the stimulation pulse duration (duty cycle), a threshold can be reached in successive steps. When the threshold is reached, an additional duration can be added to provide a level of insurance that all pacing will occur above the threshold, or it may be sufficient to hold the stimulation pulse duration at the threshold.

After each successful vagal stimulation pulse series, a determination is made regarding the difference in duration existing between the last non-effective pulse duration and the present effective pulse. That difference in duration is added to the present time. The system then senses the effectiveness of subsequent stimulation pulses and remains at the same level for the treatment period for either an unlimited duration or backs off one step in pulse duration. When the effectiveness is maintained again after a preset time window, which could be a number of beats, minutes or hours, the system backs off one decrement at a time. As soon as the effectiveness of the stimulation pulses is lost, the system keeps incrementing the duration until an effective pulse is obtained. In summary, the sensing and stimulation is a closed loop system with two feedback responses: the first response is following an effective pulse and involves gradual reduction of duration after a predetermined number of beats or a predetermined time interval; and the second response is to an ineffective pulse and is immediate with pulse duration adjustment occurring within one beat

### Stimulation Signal Generation

With continuing reference to FIG. 2, the software executed by the control unit 56 analyzes the electrocardiogram signals and other physiological characteristics from the sensor electrodes 50 to determine when stimulation is needed. As noted previously the present system can be used to stimulate other physiology, such as the brain for treatment of Parkinson's disease or obsessive/compulsive disorder, muscles, the spine, the gastro/intestinal tract, the pancreas, and the sacral nerve, to name a few examples, in which case the sensor electrodes 50 detect physiological characteristics associated with those regions. When stimulation is required the control unit 56 issues a command to the stimulation signal generator 61 which controls timing, shape and duration of the stimulation pulses. The stimulation pattern for pacing tends to be a 1 to 5 volt pulse or a pulse complex at the desired heart rate, while vagal or nerve stimulation in general requires a 10 msec to 10 second burst of 20 Hz to 200 Hz pulses each at 10 to 30 volts. The latter would require substantially more energy than a conventional pacemaker battery can provide, and a more efficient method is needed such as described here, to make operation from an implanted battery a practical proposition.

The voltage intensifier 58 preferably is a “flying capacitor” inverter that charges and discharges in a manner that essentially doubles or quadruples the battery or supply voltage. This type of device has been used in integrated circuits for local generation of additional voltage levels from a single supply. FIGS. 3 and 4 respectively illustrate a voltage doubler stage 100 and an inverter stage 102 of the voltage intensifier 58. In the doubler stage 100 of FIG. 3, a pair of switches S1 and S2 are operated by a square wave signal from a generator 104 to alternately charge and discharge an input capacitor 106 with the input voltage VIN. When the switches S1 and S2 are positioned as shown, the input capacitor 106 is charge by the input voltage VIN. During the discharge part of the switching cycle, the voltage across the input capacitor 106 adds to the voltage already across an output capacitor 108, that is connected between the output terminals of the doubler stage 100 to produce an output voltage VOUT that is twice the input voltage. In the inverter stage 102 of FIG. 4, a second pair of switches S3 and S4 are operated by a square wave signal from the generator 104 to alternately charge and discharge an input capacitor 110 with the input voltage VIN to the inverter. During the discharge part of the switch cycle of this circuit, the voltage on the input capacitor 110 is applied across the output capacitor 112 and the output terminals in a manner that inverts the polarity of the output voltage VOUT with respect to the input voltage VIN. A doubler stage 100 and an inverter stage 102 can be connected in series to produce an increased inverted output voltage with four times the battery voltage with the potential for positive and or negative swings equal to four times the battery voltage, for a total peak-to-peak amplitude of eight times the battery voltage. This voltage can be applied to apply stimulation electrodes 60. Various numbers of doubler stages 100 can be cascaded in series to increase the voltage from the battery 53 or radio frequency transceiver 54 to the desired stimulation output voltage. The number of doubler stages may be switchable in response to control signals from the control unit 56 thereby enabling the voltage to be increase by different powers of two and inverted without use of inductors or transformers. More that one pair of stimulation electrode can be provided at different locations on the heart. In that case a switch circuit controlled by the control unit selects which pair pf electrodes receive a given stimulation pulses to stimulate a particular region of the heart.

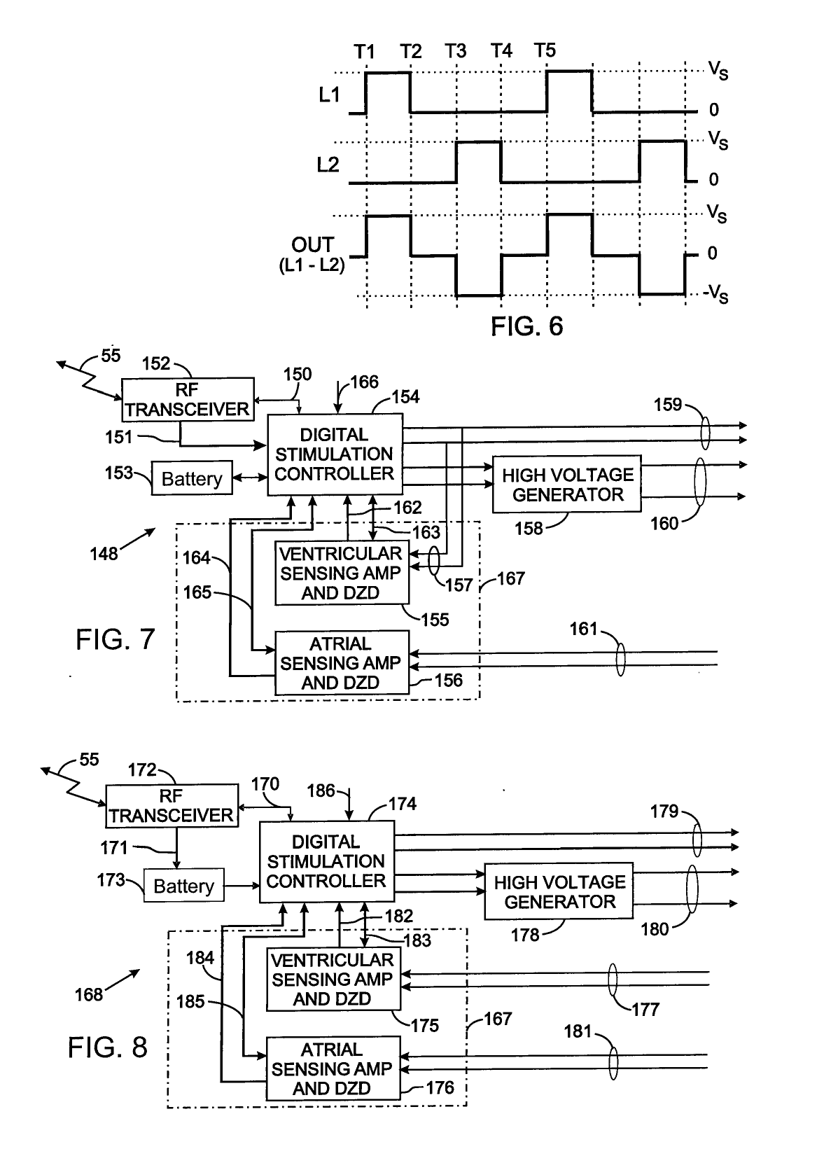

FIG. 6 depicts the output signals from the stimulation signal generator 61 or voltage intensifier 58 and present a pair of output electrodes 57 or 60, respectively. As depicted in the bottom signal waveform, the peak to peak amplitude of the stimulation voltage also can be doubled by bipolar mode operation since the circuit is not externally grounded. This is accomplished without using transformers, inverters or converters. As an example, for unipolar operation as depicted by the upper two waveforms in FIG. 6 and by the circuit in FIG. 5, one output line L1 is always kept at a zero level and another output line L2 is switched between the zero level and a maximum voltage Vs, which is the supply voltage 122. That switching is accomplished by a switch 121 that is electrically operated by a control signal 120. The resultant unipolar signal can be intensified by a voltage doubler 126, which contains the circuit 100. The output voltage 127 has a swing equal to twice the supply voltage 122 with respect to ground 124.

FIG. 6 depicts bipolar operation in which both output lines L1 and L2 are outputs from stimulation signal generator 61. Each individual signal on these output lines is unipolar as depicted in the top two waveforms, but by connecting the stimulation electrodes to these output lines without using a common ground, allows the resultant output signal in the lower waveform to be equal to the difference between the signals on output lines L1 and L2. This creates a bipolar output, which is not ground referenced and which carries a maximum relative amplitude of Vs positive or Vs negative, for a total swing of 2 Vs. An intensifier 100 can be added to further increase the amplitude.

The waveforms in FIG. 6 show how this can be accomplished. Initially at T0 both output lines L1 and L2 are connected to the supply or battery negative terminal which is arbitrarily defined as the reference zero volt level V0. At time T1, the output line L1 is switched to Vs at the positive battery terminal, while output line L2 remains connected to the negative terminal, thereby rendering L1 positive with respect to L2 by Vs. Then at time T2, output line L1 is switched to the negative terminal which returns both lines to V0 and voltage difference is equal to zero. Next output line L2 is switched to the positive terminal Vs at time T3 while output line L1 remains connected to the negative terminal V0, thereby rendering line L1 negative with respect to line L2 by Vs. At time T4 both output lines are connected to the negative terminal, making the difference equal to zero again. The switching pattern repeats successively beginning at time T5. The switching produces a waveform designated OUT, which equals L1-L2, across the two output lines and the peak to peak voltage is twice the supply voltage Vs.

### Medical Device Configuration

Having described a general embodiment to carry out the invention, a preferred embodiment is described next. It should be noted that the preferred embodiments have multiple modules, each of which are individually designed for highly efficient operation by minimizing energy losses.

Accordingly, the stimulator 148 shown in FIG. 7 comprises a low impedance power supply 149, that supplies energy to a digital stimulation controller 154. The digital stimulation controller 154 govern production of the stimulation signal with a digital output delivered to the stimulation site. The controller also operates a sensing unit 167 that has electrical sensing devices 155 and 156 which do not have external grounding. The ventricular sensing amplifier and DZD 155 has inputs connected to electrodes 159, the atrial sensing amplifier and DZD 156 has inputs connected to electrodes 161 and the digital stimulation controller 154 are connected to the animal's tissue through a very low input impedance lead assembly with a plurality of dynamically programmable electrodes. Purpose specific segmented waveforms are delivered to the electrodes by the digital stimulation controller. The digital stimulation controller 154 delivers segmented digital waveforms whose voltage amplitude is chosen such that it is close to the desired output voltage. A device capture threshold is managed by modifying the duration of the output waveform, thereby minimizing energy losses at the output stage. The segmented, stimulation waveforms may pass through a voltage intensifier stage or hi-voltage generator 158 based on a specific purpose. As an example of an application requiring voltage intensification or a high voltage generator stage, an atrial defibrillation device may require a high voltage (10-30 V) at a 20 to 200 Hz stimulation frequency. As an example for an application that does not require voltage intensification, a pacing device to treat bradycardia may need a low voltage (2-5 volts) and stimulation low rate (40-120 BPM), equivalent to a frequency of (0.67 Hz to 3 Hz). The high voltage generator 158 is connected to the target stimulation site by means of a lead assembly with a plurality of electrodes 160 that are shared with the output 157 from the digital stimulation controller 154.

FIG. 8 depicts essentially the same configuration of a stimulator 168 as in FIG. 7, but differs in that the hi-voltage generator output electrodes 180 are independent of the electrodes 177 coupled to the ventricular sensing amplifier and DZD 155, in the event that the optimum pacing site differs in location from the hi-voltage stimulation site.

The stimulators 148 and 168 in FIGS. 7 and 8 also sense a physiological characteristic of the animal and send related data via a wireless signal. The sensing device has no external ground and is therefore practically immune from noise sources that are inevitable in externally grounded devices. The output line 162 of the ventricular sensing amplifier and DZD 155 and the output line 164 of the atrial sensing amplifier and DZD 156 result from analysis by a derivative zero detector (DZD) shown in FIG. 17B or 18B). The analysis further discriminates between noise 711 (FIG. 18A) from biological signals. The stimulation may be further adapted based on the analysis performed by the digital stimulation controller 154 to optimize stimulations.

These stimulators 148 and 168 have a capability to provide stimulation for two or more clinical purposes simultaneously. For example, the medical device 10 can be configured to provide concurrent treatment for atrial fibrillation and backup pacing to increase the heart rate in cases where the heart rate falls below a predetermined rate. As another example, the medical device 10 can be configured to provide concurrent atrial defibrillation and cardiac resynchronization therapy.

In the subsequent paragraphs, each module of the stimulators 148 and 168 is described in detail.

### Power Supply:

The medical device 10 periodically receives a radio frequency signal 55 from a power source that in outside the animal. For example, the animal may wear of carry such a power source. That RF signal may include data and programming instructions which the RF transceiver 152 or 172 sends via connection 150 or 170 to the digital stimulation controller 154. The RF transceiver 152 or 172 also derives electrical power from a received RF signal 55 and distributes that power via lines 151 or 171 to the modules of the stimulator 148 or 168.

The power supply 149, alternatively or in addition to the RF transceiver 152 power supply, has battery 153 such as a “can” type battery, a piezoelectric device, thermal energy source, mechanical energy source or chemical energy source. In some embodiments, two or more of the energy sources e.g. 152 and 153 depicted in FIG. 7 and 172 and 173 depicted in FIG. 8, may be combined to supply power to the medical device 10. In any case, it is very important to have the energy source with low source impedance to minimize energy loss within the source itself.

### Digital Stimulation Controller

With reference to the two stimulators 148 and 168 in FIGS. 7 ad 8, the digital stimulation controllers 154 and 174 store operational parameters for use in controlling the stimulator. Preferably, the digital stimulation controller 154 or 174 comprises a conventional microcomputer that has analog and digital input/output circuits and an internal memory that stores a software control program and data gathered and used by that program.

The digital stimulation controllers 154 and 174 also receive data from a plurality of sensor electrodes 161 and 159 in FIG. 7 and sensor electrodes 181 and 177 in FIG. 8, that detect electrical activity of the organ of interest, such as conventional electrocardiogram signals. The sensor signals are utilized to determine when a stimulation therapy should occur. Additional sensors for other physiological characteristics, such as temperature, blood pressure or blood flow, may be provided and connected via input 166 and 186 to the respective digital stimulation controller 154 or 174. The digital stimulation controller stores a histogram of pacing data related to usage of the medical device and other information which can be communicated to a device external to the patient.

### Segmented Waveforms and Low Impedance Lead:

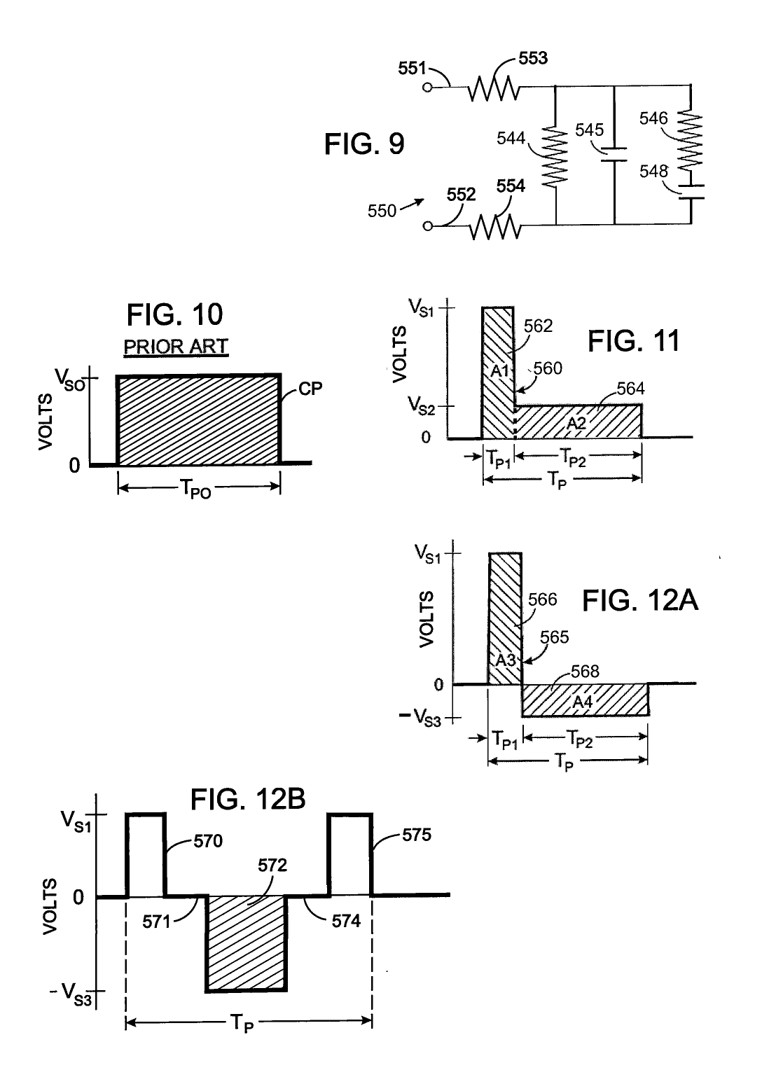

A novel ultra low resistance pacing lead circuit may be used with the present stimulators 148 and 168. In FIG. 9, the pacing lead circuit 550 has first and second conductors 551 and 552, the combined resistance of which less than 100 ohms, and preferably is less than ten ohms. Specifically, each conductors 551 and 552 has a aggregate resistance 553 and 554, respectively. The electrical characteristics of the tissue being stimulated are modeled as an equivalent resistance 546 in series with an equivalent capacitance 548, that are in parallel with the capacitance 545 and tissue leakage resistance 544. The dominant time constant is formed by the aggregate lead resistance 553 and 554 and the tissue capacitance 545, the pacing lead circuit 550 has a significantly smaller primary RC time constant formed by (resistances 553+554) time capacitance 545, which consequently allows for faster rise and fall times of the stimulation pulse. The distributed tissue resistance 546 and capacitance 548 have less impact on the effective high speed pulse, but do affect the pulse propagation speed, which is not significantly different between low impedance leads as described here and conventional high impedance leads (e.g. having impedances greater than 200 ohms). The primary effect is that of faster stimulation at the stimulation site with less energy.

Upon activation of the stimulator 148, the digital stimulation controller 154 in FIG. 7 executes a software program that based on heart rate determines when and how to stimulate the animal's tissue. The digital stimulation controller 154 receives signals from the sensor electrodes 159 and 161 that indicate the electrical activity of the heart and analyzes those signals to detect irregular or abnormal cardiac activity. When pacing is needed, the stimulator 148 applies electrical voltage pulses to either electrodes 159 or 160 in the manner described previously.

The waveform of each of those electrical voltage pulses, referred to as a composite pacing pulse, is illustrated in FIG. 11. The composite pacing pulse 560 is characterized by a first segment 562 and a second segment 564 contiguous with the first segment, and preferably immediately following the first segment as illustrated. Both the first and second segments 562 and 564 have rectangular shapes with the understanding that in actuality a rectangular pulse has leading edge that does not have an infinite slope and thus has a non-zero rise time. Similarly the trailing edge of the first segment also has a non-zero fall time. Specifically, the first segment 562 has a fast rise time (4V/μs); a duration between 0.005 ms and 0.5 ms, and preferably 0.2 ms and a similarly fast (4V/μs) fall time.

The amplitude V_{S1} of the first segment 562 is at least three times greater than the amplitude V_{S2} of the second segment 564. The second segment 564 has a significantly longer duration T_{P2}, e.g. at least three times the duration T_{P1} of the first segment 562. The integral of the first segment 562 is graphical depicted by area A1 under that segment of the pulse, and integral of the second segment 564 is depicted by area A2. Preferably, the integral of the first segment 562 is substantially equal to the integral of the second segment 564.

The amplitude of the first segment 562 of the composite pacing pulse 560 is at least three times greater than the conventional nominal amplitude V_{SO}, shown in FIG. 10, while the second segment 564 has an amplitude that is less than that nominal amplitude. The total duration T_{P} of the composite pacing pulse 560 is less than the nominal duration of the conventional pacing pulse. The sum of the integrals for the first and second segments is less than the integral of the conventional pacing pulse CP in FIG. 10, i.e. total area (A1+A2) of the composite pacing pulse 560 is less than area A0. Further note that the efficiency is gained by expending less overall energy and the clinical efficacy is gained by reducing the stimulation threshold for most of the duration of the pulse.

FIG. 12A illustrates an alternative composite pacing pulse 565 which is characterized by a fast rising, short duration, high positive amplitude first segment 566 that is substantially identical to the first segment 562 of the previously described pulse in FIG. 11. However, the first segment 566 is followed by a different second segment 568 consisting of a negative voltage with an absolute amplitude that is equal to or less than one-third the absolute amplitude of the first segment 566. The duration T_{P2} of the second segment 568 is a significantly longer than, e.g. at least three times, the duration T_{P1} of the first segment 566. Here too, the integral A3 of the first segment 566 is substantially equal to the integral A4 of the second segment 568. Consequently, the absolute sum of those integrals is less than the integral of the conventional pacing pulse CP, i.e. total area under the first and second segments (A3+A4) is less than area A0 in FIG. 10.

It should be noted that in contemplated embodiments, waveforms chosen may be biphasic or triphasic or multiphasic with pauses in between segments. An exemplary triphasic waveform is illustrated in FIG. 12B. The stimulation cycle starts with a positive lobe segment 570 having amplitude of V_{S1}. It is followed by a pause segment 571 with amplitude of 0V. The segment 572 is a negative lobe segment of amplitude-V_{S3}. Next is a pause segment 574 of 0V amplitude that is followed by a positive lobe segment 575 of amplitude V_{S1}. It should be noted that if charge balancing to e.g. avoid electrolysis at the electrode tissue interface, is required at the stimulation site, the sum of the integral of positive segments should be set equal to the sum of the integral of negative segments. The amplitude of the positive and negative segments may or may not be equal. An example sequence may have a 50 μs positive pulse segment, a 20 μs pause, 100 μs negative segment, 20 μs pause, and a 50 ms positive segment, with equal absolute amplitudes for the positive and negative segments. The notation used here for the representation of the waveform sequence is “+” for positive segments, “0” for the pause and “−” for the negative segments. This gives a sequence of +, 0, −, 0, + with a total of 100 μs positive, and 100 μs negative pulse segments. Another example sequence is 50 μs positive, 20 μs pause, 50 μs negative (+, 0, −). Yet another example of a sequence with more than 3 phase segments is +, 0, −, 0, +, 0, −, . . . It should be further noted that these segmented waveforms can be a part of a continuous stimulation regimen wherein the tissue is stimulated by the predetermined composite waveform sequence at intervals determined by the period of the stimulation frequency. In certain applications the stimulation may be command driven such that the stimulation is applied only if certain stimulation criterion that is programmed in the control circuit is met. In such cases the digital stimulation waveforms would pause for a command from the control circuit before applying a segmented, composite stimulation waveform at a tissue location.

In some embodiments, the stimulated tissue may be cardiac muscle, or a nerve such as vagal nerve or a spinal nerve, bladder, brain or spinal tissue, to name a few. As mentioned earlier, in some embodiments, traditional devices such as pacemakers and defibrillators, pacemakers for vagal stimulation for atrial fibrillation therapy, and other types of pacers for bradycardia, resynchronization, vagal stimulation for central nervous system (CNS) conditions may benefit from the segmented composite stimulation waveforms.

### Sensing Circuit:

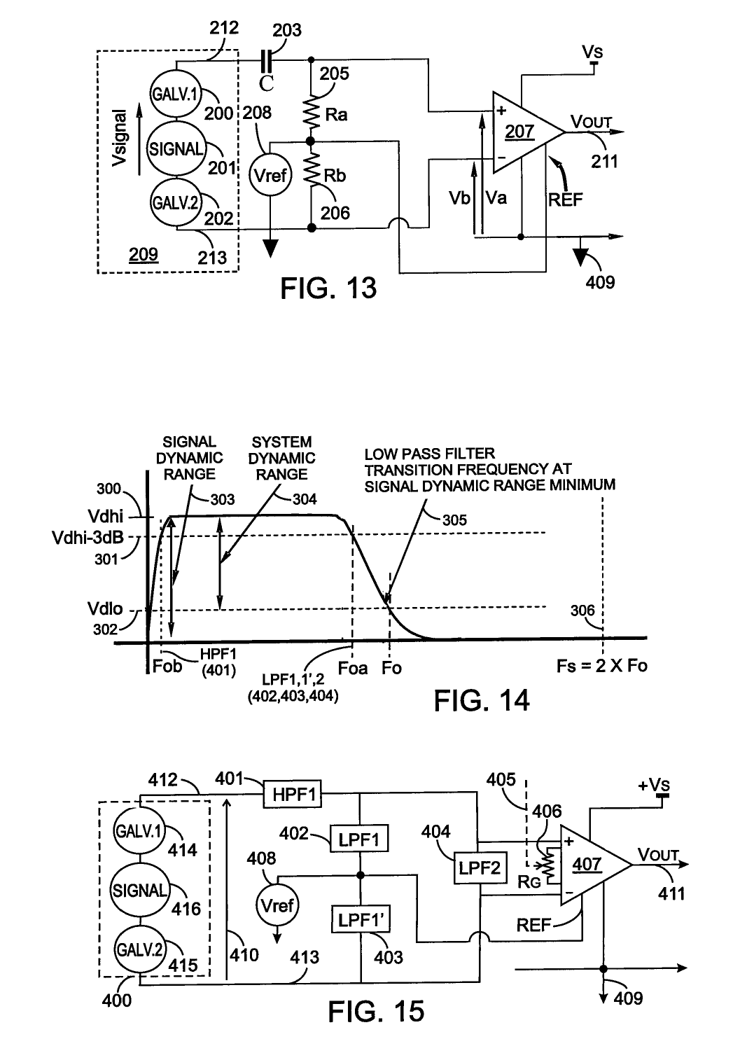

The sensing circuits 155 and 156 in FIG. 7, and sensing circuits 175 and 176 of a sensing unit 188 in FIG. 8, comprise an instrumentation amplifier circuit represented in FIG. 13, and a derivative zero transition detector shown in FIG. 18B. In the preferred embodiment, the sensing circuit is not connected to an external ground.

There are a few considerations in a practical implementation of the sensing circuit 155 and 156 in FIG. 7 or 175 and 176 FIG. 8. First, there are DC considerations. Second, there is an internal reference consideration. The third involves filtering considerations.

DC Considerations: Referring to FIG. 13, in the physiological environment 209 at the interface between electrode and tissue, a galvanic system is formed with a DC potential. If there is complete symmetry in this circuit from a first electrode 212 to a second electrode 213, then the sum V_{SIGNAL} of all the contact potentials cancel. However, if the electrode materials are dissimilar or are at different temperatures, the electrode-tissue and or the electrode-blood interface yields potentially different galvanic generator values at electrodes 212 and 213 that do not cancel. In this case, the input amplifier 207 is presented with the sum V_{SIGNAL} of the source voltage 201 of interest along with the galvanic potential difference. This galvanic components 200 and 202 are relatively static, but potentially are modulated by body or organ movement, as the electrode may wander between touching the blood vessel wall and the blood pool, thereby presenting a varying “DC” voltage. The variance over time is expected to be synchronous with the movement, and thus in the sub 2 Hz range, if respiratory and cardiac movements are included. Another DC issue stems from the amplifier 207 itself, which will require a DC current bias into or out of the amplifier input terminals. In MOSFET amplifiers, this “bias current” is very small, but doubles with every 10° C. in temperature rise. Also, this current can have an offset, leaving a differential current that can spoil the balance of a high impedance circuit. The solution to this problem is to provide a form of AC coupling with the electrodes 212 and 213, and a DC current path for the bias currents is offered via resistors 205 and 206.

The AC coupling capacitance 203 performs two functions. The first function is DC decoupling from the galvanic voltages, Galv.1,200 and Galv.2,202, and the second function is to form a high pass filter 401 (see FIG. 15) with a corner frequency of F_{HP}=½πRC, where R=Ra+Rb respectively resistors 205 and 206 and C is represented by capacitance 203 in FIG. 13.

The bias and offset currents are in the order of 10⁻⁹ to 10⁻⁸ A, and with input circuit path resistances of e.g. 100 kOhm, still yield 0.1 to 1.0 mV. Since source voltages are in order of 0.5-10 mV, these bias and offset voltages are not negligible. Therefore, for the stimulators 148 and 168, the amplifier specification selection should be such that these currents are low enough to allow for reasonably high input circuit resistance values in the order of 100 kOhm or better for resistors Ra 205 and Rb 206.

Appropriate selection of resistors Ra 205 and Rb 206, yields an acceptable low bias current offset voltage component (V_{offset}=I_{offset}×Ra, where Ra=Rb and I_{offset}=I_{bias-a}−I_{bias-b}), and a practical value for the filter frequency F_{HP} of the high pass filter (HPF1) 401 in FIG. 15. The traditional corner frequency range for high pass filter 401 is in the order of 0.5 Hz to 2.0 Hz, but other values can be selected depending on spectral regions of interest.

A natural feature aid in the proposed implementation is the relatively low impedance of the animal tissues involved, typically 300 to 1200 Ohm between, for example, 2 mm to 5 mm spaced electrodes. Thus, in order to create a net 1.0 mV across such an impedance, energy density of approximately 0.4 mW/m would be needed with the energy contained in the 0-1 kHz band.

Reference Considerations: In order to incorporate a floating AC coupled signal, it is desirable to provide a reference point 208 in FIG. 13. If the signal is expected to be symmetrical, a V_{ref}=Vs/2 can be selected, thus allowing Vₒᵤₜ to swing between ground and Vₛ, with a rest point at V_{ref}, This reference input is provided to the output stage of the amplifier 207. Commercially available instrumentation amplifiers have a provision to receive reference input for the amplifier output stage. The original input signal now can be presented at the output as: Vₒᵤₜ=V_{signal}×Gain×F, where F is a high pass filter function.

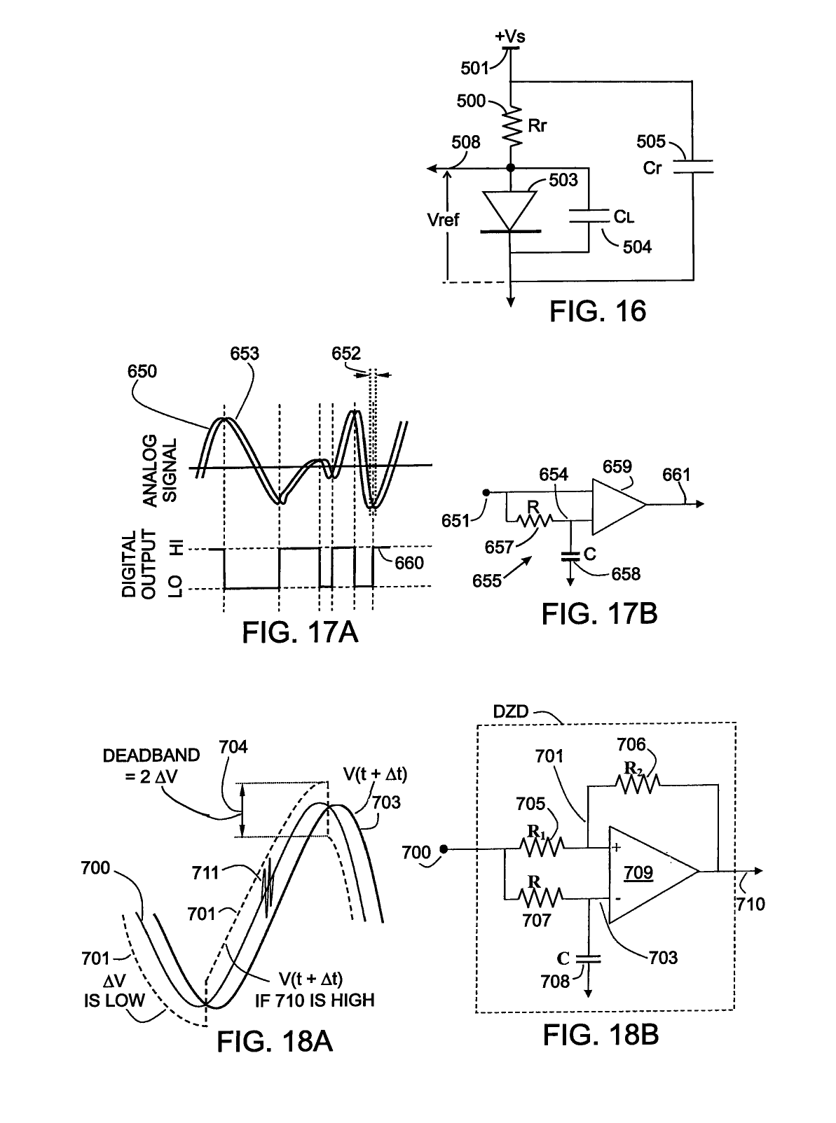

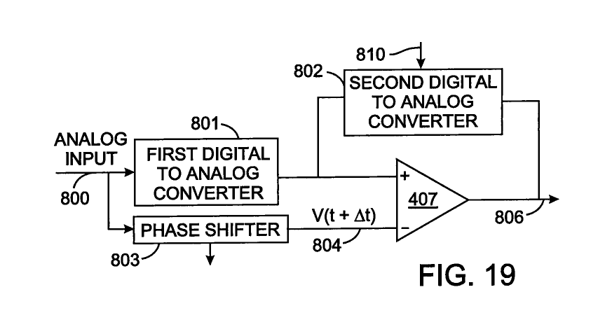

Additional details for the internal reference 408 in FIG. 15 are provided in FIG. 16. A reference voltage of 1.2V to 1.4V is achieved using a Gallium-Arsenic light emitting diode (LED) 503 that is supplied via resistance Rr 500 and decoupled from noise by capacitors Cr 505 and CL 504. Two factors enable an LED 503 to be used as a stable reference voltage 508. First, the electronics module containing signal amplifier (FIG. 15) and detector (FIG. 19) as a part is in an intravascular environment, wherein the blood pool provides an electromagnetic interference (EMI) shielding function. Second, the thermal properties of this environment are relatively constant at the internal body temperature. Thus, it can be shown from the fundamental considerations that the voltage drop across the LED 508 remains sufficiently constant at relatively constant temperature.

Filtering Considerations: Referring to FIG. 14, if there is no meaningful information contained in the filtered out band above Fo 305, there will not be any adverse issues with the filtering approach. In practical applications, that is however rarely the case because the frequency chosen for Fo 305 tends to be relatively low (<100 Hz) in the interest of EMI suppression. Since important information is contained in those frequency bands, the current implementation is tailored to include the entire band from 10 Hz to 250 Hz. For robustness reasons even a wider range of frequencies (e.g., 2 Hz-500 Hz) is used. With this consideration, the fast rise time of the sinus node signals containing high frequency content in the 100-250 Hz range can be easily accommodated in their pristine form. Additionally, by including these frequency components, the natural physiological signals can be easily distinguished from background signals, such as noise, voluntary and involuntary muscle movement etc. In order to provide sufficient signal for analysis, sufficient resolution must be available to the detection algorithm contained within the computer (154, FIG. 7). The system dynamic range 304 is defined by the maximum amplitude Vdhi 300 and the noise floor Vdlo 302. Usually a system resolution of ten bits (1/1024 steps) or twelve bits (1/4096 steps) is desired, although it is possible to get basic results with even eight bits (1/256 steps) resolution, with the exception that lower resolution system have reduced ability to distinguish detailed features in the signal, as may be desirable for signature analysis.

The details of this aspect of invention are disclosed in FIGS. 14 and 15. Physiological environment 400 is shown to contain the galvanic voltages 414 and 415 formed at the tissue electrode intersections of two electrodes in an exemplary embodiment. The biological signal source that would be sensed is shown as the sum of signals 410 with an associated signal voltage 416. The source may also have associated series source impedance (Z_{source}), which is not shown as it will be very low as compared to the overall system input impedance looking into electrodes 412 and 413.

Between the biological environment 400 and the signal amplifier 407 in FIG. 15, at last three filters are provided to perform various functions. The first is a high pass filter 401 that essentially blocks DC and low frequencies up to a prespecified cut-off (e.g., 2.0 Hz). This high pass filter 401 consists of passive elements with capacitance and resistance, where resistance may be obtained by a combination of resistors, and source impedance in series. The second filter suppresses common mode noise by providing a suitable first low pass filter (LPF1) 402. This first low pass filter 402 consists of passive elements C and R and their symmetrical counter parts in low pass filter (LPF1′) 403.

The third filter 404 rejects high frequency noise signals using a low pass filter (LPF2) which consists of passive elements capacitor and resistors in series. Electromagnetic broadband ambient noise from appliances and other equipment could swamp the input circuit and consume dynamic range. Such ambient noise needs to be filtered out. In one embodiment, low pass filter LPF2 404 with a cut-off at 1 kHz frequency is selected since the Electromagnetic noise is broadband, but its energy is rather low below 1 kHz and can be effectively filtered out.

Other Considerations: For ECG signals obtained by direct connection to the cardiac venous vessel wall or muscle tissue, the signal path between the two or more input electrodes 412 and 413 should be constructed such as to avoid forming an electromagnetic pickup loop, for example, by twisting the lead and or wire pairs, which would effectively help to cancel electromagnetic noise pick-up. Therefore, symmetrical layouts are favored.

In summary, as noted above, the absence of a traditional ground in the present stimulators 148 and 168 is a significant departure from the prior stimulation devices and has obviated the need for notch filtering and other kinds of signal degrading processes. Another important aspect of the invention as already mentioned is the use passive filtering at the front end, before any active components are involved. As a result, physiological signals without any degradation are obtained. Finally, if used in “can” type implanted devices that have a “can” type metal housing, the sensing electrodes 412 and 413 do not form circuit including the “can” as used in prior stimulation devices, since the “can” is in contact with patient's tissues and form loops between itself and electrodes 412 and 413, which is not desirable and cause for noise collection.

Signal Detector: The two sensing amp and DZD's 155 and 156 in FIG. 7, have stimulation electrode inputs connected to a variable gain instrumentation amplifier 407 shown in FIG. 15. That variable gain instrumentation amplifier 407 has an output signal 411 coupled to an analog input line 162 or 164 of the digital stimulation controller 154 in FIG. 7. The output signal 411 from the instrumentation amplifier 407 also is applied as an input signal 650 to an input 651 of a derivative zero transition detector (DZD) 655 in FIG. 17B. The DZD 655 performs signal transition detection and provides an output signal 660 on line 661 to the digital stimulation controller 154 (FIG. 7) that indicates of time events in the sensed physiological data signal.

In a preferred embodiment, the signal detector comprises a signal transition detector followed by an event classifier contained within the software of the digital stimulation controller 154. The derivative zero transition detector 655 as shown in FIG. 17B includes a comparator 659, which is presented with the signal 650 in FIG. 17 at input 651 and a time shifted copy of the signal 653 (for example, a composite sinusoidal waveform) at another input 654, wherein the comparator identifies features in the signal that are distinguished by having a local zero derivative representing the change of direction of the signal amplitude. The output signal 660 at the amplifier's output line 661 is a digital representation indicating signal direction change and time between these events.

The derivative zero transition detector 655 can be implemented using conventional operational amplifiers for frequencies less than 200-400 Hz. However, for higher frequencies, comparator operational amplifiers are preferred to provide a digital output signal with well-defined slopes. The method is sensitive to the time delay value 652, which will separate the signals in time. There are a number of conditions to consider in choosing the time delay value. Which could be implemented by varying the resistance of 657. It should prevent setting off events from small random noise amplitudes. It could be set to exclude certain portions of the cardiac signal time sequence. For example, when a good QRS signal is detected, a larger delay can be chosen.

In FIGS. 19A and 19B, it can be seen that the waveform amplitude transition threshold (deadband 704) required to trip the comparator is a function of the associated hysteresis of the circuit, and the open loop gain of the comparator. The hysteresis amount ΔV is a function of the deadband that can be chosen based on the component selection. The resistors R₁ 705 and R₂ 706 are chosen such that their ratio approximates the desired hysteresis. The components resistor 707 and capacitor 708 determine the time constant of the delay. The threshold required to switch states is a function of the gain and slew rate of the comparator or operational amplifier 709 at the frequencies of interest. Typically the gain roll off rate is 20 dB per decade from 1 kHz onward. With such a roll off point, a 105 dB gain at 1 kHz reduces to a gain of 65 dB at 100 kHz. The slew rate is the maximum rate by which the output 710 can change states. For example, a 1V/msec slew rate would require at least five milliseconds to go from 0 to 5 volts, regardless how hard the input is being overdriven.

The output 710 of the detector is a transformed signal that is discrete. It should be noted that this technique is immune to the variations in dynamic range of the input signal unlike traditional methods. The discrete signal can be advantageously used for signal classification.

FIG. 19 shows the an enhanced variant of the DZD depicted in FIG. 18B. Here the signal at the input terminal 800 is connected to a first digital to analog (D/A) converter 801 that produces an output 804 which is applied to the non-inverting input of a comparator 807. The inverting input of the comparator 807 receives an output from a constant amplitude adjustable phase shifter 803 that receives the signal from the input terminal 800. The output 806 of the comparator 807 is coupled by a second D/A converter 802 to the non-inverting input. The second D/A converter 802 is controlled by a signal on line 810. The two D/A converters 801 and 802 allow feature based real time feedback for training the system on the signal and enhancing a particular feature of interest as for cardiac rhythm, fibrillation, lead breakage, EMI, but also for training a voice recognition system on specific speakers.

For example, the DZD depicted in FIG. 19 in conjunction with software executed by the digital stimulation controller 154 can determine the heart rate and use this information in an algorithm for pacing a patient's heart. The heart rate detection is based on the number of transitions counted over a predefined time interval. If the heart rate goes out of range for a given length of time and the frequency of the transitions remain in the non-fibrillation range, cardiac pacing can be initiated to pace the patient's heart. When the transition frequency indicates atrial fibrillation, vagal nerve stimulation for the purpose of lowering the heart rate can be initiated.

Controlling the sensing circuit: Referring again to FIG. 15, when stimulation is occurring, the instrumentation amplifier has low gain (0.1× or lower) to avoid saturation. When stimulation is inactive (high impedance across stimulation electrodes) as occurs between heart beats, the instrumentation amplifier has a normal gain (100×−200×) to sense physiological characteristics. The gain change is programmably achieved by commands from the digital stimulation controller 154 sent via line 163 or 165 (FIG. 7) to a control port 405 of the instrumentation amplifier 407. The low gain setting allows measurement of the tissue and electrode interface impedance by using the known stimulation pulse duration and amplitude as a known source and the system impedance as known impedance. From taken timed samples of the sensed voltage and the known impedances, the tissue and electrode interface impedance can be determined. This information can also be logged over time to monitor physiological changes that may occur.

For stimulation verification, the digital stimulation controller 154 analyzes the sensed parameters to calculate the actual heart rate to determine whether the heart is pacing at the desired rate in response to the stimulation. If the heart is pacing at the desired rate, the digital stimulation controller 154 can decrease the stimulation energy in steps until stimulation is no longer effective. The stimulation energy then is increased until the desired rate is achieved. Energy reduction can be accomplished at least in two ways: (1) preferably, the duty cycle is reduced to linearly decrease that amount of energy dissipated in the tissue, or (2) the voltage amplitude is reduced in situations where energy dissipation might vary non-linearly because the tissue/electrode interface is unknown.

The stimulation is controlled by a functionally closed feedback loop. When stimulation commences, the sensed signal waveform can show a physiological response confirming effectiveness of that stimulation pulse. By stepwise increasing the stimulation pulse duration (duty cycle), a threshold can be reached in successive steps. When the threshold is reached, an additional duration can be added to provide a level of insurance that all pacing will occur above the threshold, or it may be sufficient to hold the stimulation pulse duration at the threshold.

After each successful stimulation pulse, a determination is made regarding the difference in duration existing between the last non-effective pulse and the present effective pulse. That difference in duration is added to the present time. The system then senses the effectiveness of subsequent stimulation pulses and remains at the same level for either an unlimited duration or backs off one step in pulse duration. When the effectiveness is maintained again after a preset time window, which could be a number of beats, minutes or hours, the system backs off one decrement at a time. As soon as the effectiveness of the stimulation pulses is lost, the system keeps incrementing the duration until an effective pulse is obtained. In summary, the sensing and stimulation is a closed loop system with two feedback responses: the first response is following an effective pulse and involves gradual reduction of duration after a predetermined number of beats or a predetermined time interval; and the second response is to an ineffective pulse and is immediate with pulse duration adjustment occurring within one beat.

### Exemplary Clinical Applications:

Having described the complete stimulation system, an exemplary clinical application can now be described to illustrate the utility of the invention.

Application 1: Vagal stimulation to treat atrial fibrillation with backup pacing: With reference to FIG. 1, atrial fibrillation rate control is carried out using stimulation of the vagus nerve near the proximal coronary sinus (CS) 18 or from the inferior vena cava (IVC) 21 at the entry into the right atrium 16, or from the superior vena cava (SVC) at the entry 12 into the right atrium. The literature on atrial fibrillation has demonstrated that it is a clinical possibility. However, existing pacers cannot realistically perform atrial fibrillation treatment because of the energy that would be required for such stimulation with a continuous 20-200 Hz, 20V waveform. Using the current inventive system depicted in FIG. 7, an efficient digital waveform based stimulation protocol consuming less energy makes it practical to use a conventional pacemaker battery 153 or use energy from the RF transceiver 152 to power the stimulator 23. Additionally, segmented waveforms in conjunction with the use of a low impedance lead/system and the flying capacitor voltage intensifier FIG. 3 and FIG. 4 can be used to achieve the desired therapy. In one embodiment, atrial fibrillation treatment can be achieved in a “can housing” such as the one used for a traditional pacemaker. The modules of the stimulation described earlier enable a compact implementation of this novel therapeutic device that can be implanted in a similar fashion as a traditional pacemaker.

Accordingly medical device 10 for vagal stimulation to treat atrial fibrillation using energy efficient digital stimulation system comprises two or more electrodes that are programmably selectable; waveforms that are programmably selectable; and the technique optimized to avoid ventricular fibrillation. The apparatus further comprises a backup pacemaker to raise the heart rate if it falls below a predetermined threshold during atrial fibrillation treatment.

During a treatment procedure, stimulation electrodes 24 are placed at locations near the vagal nerve 14, 17, such that one or more electrodes from a plurality of electrodes are programmably selected for optimal vagal stimulation. The stimulation waveforms are programmed with respect to shape, duration and duty cycle for maximizing energy conservation and minimizing stimulation sensation to patient. The atrial fibrillation sensing and stimulation further involves sensing right atrium (RA) 16 and right ventricle (RV) 15 or left ventricle (LV) 22 and detecting when RA rate is faster than RV or LV rate. This detection may be done by the DZD detector earlier.

Programmable parameter initiates vagal stimulation based on RV/LV heart rate. By setting an upper heart rate limit, vagal stimulation is employed when the limit is exceeded. It should be noted that in patients with known chronic atrial fibrillation, an atrial electrode may not be necessary and just the ventricular rate sensing may be used. This is also the case in other supraventricular tachycardias as well.

Ensuring patient safety during vagal stimulation: Atrial fibrillation (Afib) treatment is characterized by a high voltage stimulation of the vagus nerve 14, 17 by means of a stimulation lead placement in the proximal coronary sinus (CS) 18 location at 20-200 Hz. During this stimulation particular care must be exercised to ensure that the LV 22 is not inadvertently being paced from the CS 18 location. This feature is needed because high voltage rapid stimulation (such as 20-200 Hz stimulation) of the ventricle may induce a rapid life threatening ventricular arrhythmia. It is, therefore, desirable to confirm prior to such stimulation of the vagal nerve that the electrode has not unintentionally moved where the ventricle might be stimulated.

Safety can be ensured in several ways including controlling the frequency and rate of stimulation and real-time analysis of results of stimulation. From the stimulation control approach, high voltage pacing at lower heart rates that are unlikely to induce life threatening ventricular fibrillation may be used to confirm that the ventricle is not being stimulated. In an analysis-based approach, comparing morphology of electrograms from the distal CS (LV) before and during pacing and noting that the morphology would not change if the LV 22 is not being paced. Furthermore the heart rate detected from the LV would not be the same as the paced rate. A preferred method may utilize both stimulation and analysis approaches, wherein the heart is paced at rates near the ventricular rate prior to the vagal stimulation, and a comparison of the electrocardiogram before and after such pacing is performed. The comparison results would show no change in the morphology of the electrogram if the ventricle were not being stimulated. Moreover, if pacing were performed at a rate slightly faster than the heart rate prior to vagal stimulation, the heart rate would not change if there was no stimulation or “capture” of the ventricular muscle.

Application 2: Backup LV pacing during vagal stimulation: Back up LV pacing is performed if the heart rate becomes very slow, resultant from vagal stimulation. In order to protect the patient in case the heart rate is excessively slowed beyond a programmable rate, e.g. 60 beats/min, demand pacing (pacing which occurs when a predetermined time interval passes with no electrical activity) would occur and continue until the intrinsic heart rate exceeds the programmed lower limit rate. Note that the above-mentioned vagal stimulation with LV bradycardia pacing as a backup may also be used to reduce need for medication.

As the above exemplary clinical application illustrates, the high efficiency digital stimulation device enables a number of functionalities that improves upon existing techniques. By way of examples, one embodiment of such applications involves bradycardia pacing treatment from an implanted pacemaker “can housing.” In this application, the high efficiency system provides longer battery life and fewer battery changes resulting in less frequent surgeries. In another embodiment of clinical applications, a high efficiency device can improve the battery utilization since resynchronization pacing for congestive heart failure requires pacing devices to be used continuously. In addition, demand for power is higher for this application when compared to traditional bradycardia pacing since more sites need to be stimulated including both ventricles as well as the atrium. Again referring back to atrial fibrillation treatment, high efficiency may permit therapies now limited because of the relative inefficiency of prior art. As yet another example, in an intravascular stimulation system a higher efficiency permits longer times between recharging cycles and smaller intravascular storage components.

In addition to the energy efficiency, robust sensing described in the inventive modules provides further advantages beyond the systems described herein. For example, in bradycardia pacing robust sensing translates to less inhibition or inappropriate tracking from internal and external electromagnetic interference. In another example, implantable cardioverter defibrillators robust sensing module may lessen chances of inappropriate shock therapy from EM interference or internal noise such as those that occur from lead fractures and header connections.

Application 3: Cardiac resynchronization therapy with vagal stimulation: The efficient stimulation framework described herein is ideally suited for treating the CHF patients with or without AV synchrony. The logic to perform the actions needed for therapy may be implemented as firmware or software in the controller.

Patients with AV synchrony: In prior art systems, cardiac pacing is performed if the pacing AV interval is less than the intrinsic AV interval. Therefore, optimum ventricular filling may not occur and patient may not be receiving maximum benefit. Furthermore, in those systems it becomes a tradeoff between allowing CRT pacing to occur, and allowing maximum filling to occur.

The device described herein can slow the heart rate and prolong the AV interval by the stimulation of vagus nerve as described earlier. For example, in the transvascular application, vagal stimulation may be carried out from jugular vein. The treatment may be provided by slowing ventricular rate to permit CRT and prolonging AV interval to allow greater filling time.

Patients without AV synchrony: In the case of patients without AV synchrony, for example people with atrial fibrillation, the heart rate can be slowed down by vagal stimulation. In order to slow the AV node, the proximal part of the coronary sinus, for example, may be used for the intravascular stimulation. Traditional cardiac resynchronization therapy can be more easily carried out following the vagal stimulation, as the pacemaker may no longer be inhibited.

As described before, in both cases, traditional cardiac resynchronization therapy can be more easily carried out following the vagal stimulation, as the pacemaker may no longer be inhibited. During this process, the CHF treatment may or may not involve the right ventricle. The site of the vagal stimulation can be chosen based on the heart node that has to be slowed down. In order to slow the AV node, the proximal part of the coronary sinus, for example, may be used for the intravascular stimulation. On the other hand, if slowing of SA node is required, a site in the carotid artery can be stimulated. In addition, the present application allows one to stimulate left atrium and left and or right ventricle to further improve mitral insufficiency by reducing the intra-atrial delay in dilated hearts.

Application 4: Ventricular fibrillation/ventricular tachycardia (VF/VT) detection: This application is described for systems that may be a single lead system or a two lead system.

A single lead VF/VT detection in current systems is based on: i) heart rate; or ii) heart rate and comparison of ventricular electrogram morphology of a predetermined template electrogram to the electrogram during the rapid rhythm. Similar electrograms imply the rhythm is not of ventricular origin and a treatment is withheld. The above algorithm has significant deficiencies because: i) in the detection zones for very rapid rhythm such as VF morphology algorithms are usually not employed for concern of missing a life threatening rhythm; and ii) during some rapid atrial rhythms (such as atrial fibrillation) the morphology of the ventricular electrogram changes for physiologic reasons, for example, due to ventricular aberrancy and the morphology algorithms will mistakenly identify this as ventricular in origin and unnecessarily shock the patient.

Two-lead detection systems employ a sensing lead in the atrium and the ventricle. In addition to the detection schemes noted above for a one-lead system in the ventricle, a two-lead system has the additional advantage of comparing the heart rate in the atrium and the ventricle, and chamber sequence activation. Generally the chamber with the higher rate is the chamber of origin of the rapid rhythm. Therefore, if the ventricular rate is faster then therapy is given, but if the atrial rate is faster therapy is withheld. While this is an improvement over a one-lead system, such algorithms are again not employed in very rapid heart rate detection zones, for example, VF detection zone. More importantly, even in lower heart rate detection zones, for example, a VT detection zones, this system is suboptimal when rapid rhythms occur in both chambers at the same time. Exemplary arrhythmias of this type are atrial fibrillation and atrial flutter, which are common abnormal rhythms. During such rhythms a coincident ventricular tachycardia may be missed because the atrial rate is likely to be faster than most all ventricular rhythms. In such scenarios misdiagnoses are known to occur. Even such algorithms as looking for heart rate stability, which is frequently a sign of VT, have limitations as stability of heart rate can also occur with atrial tachycardia, and heart rate variability can occur with ventricular tachycardia.

To summarize, all the present arrhythmia detectors are based on morphology or relative rates in the cardiac chambers. These methods have inherent limitations and, in clinical practice, have not eliminated unnecessary therapies. Many patients receive unnecessary therapy for rapid atrial rhythms such as atrial fibrillation because the detector cannot easily discriminate where the rhythm originates. Moreover, if the rate is very rapid, existing detectors are designed to over estimate abnormal rhythms rather than missing a serious rhythm. Therefore, there is a need for a detector that can discriminate VT from supra ventricular tachycardia (SVT).

With the present high efficiency stimulation framework, detection of a rapid ventricular rhythm is followed by vagal stimulation as described. Subsequently, if we sense that the heart rhythm is slowed by the vagal stimulation then it most likely that the rapid ventricular rhythm has originated in the atria and is not life threatening. In such cases, therapy can be avoided. Moreover, if the heart rhythm is slowed, the rate will likely drop out of the detection zone, for example, the programmed heart rate. Note that vagal stimulation does not slow VT or VF. It slows conduction in the AV node and thus slows the ventricular rate of atrial rhythms originating above the AV node.

In general, the proposed method is applicable with either pacing alone, with or without cardiac resynchronization therapy (CRT), with or without ICD, to distinguish rapid atrial fibrillations or other supra ventricular tachycardias (SVT's) from VT/VF by the application of vagal stimulation to cause slowing of the ventricular rate. When used with an ICD, this method will reduce unnecessary shocks.

The foregoing description was primarily directed to a preferred embodiment of the invention. Although some attention was given to various alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

1. An implantable digital stimulation system for atrial fibrillation treatment by electrical stimulation of a vagal nerve in a patient, the implantable digital stimulation system comprising:
a plurality of electrodes at one or more intravascular locations in proximity to a vagal nerve inside the patient; and
an implantable stimulator comprising:
(a) a control unit for controlling the electrical stimulation by programmable selection of at least some of the plurality of electrodes and by programmable selection of one of a plurality of stimulation waveforms, and
(b) a stimulation signal generator connected to the control unit and producing stimulation signals that have segmented waveforms that are programmably selectable by the control unit;
(c) an implantable sensing unit with internal reference to monitor a heart rate of the patient during the stimulation treatment.

2. The implantable digital stimulation system as recited in claim 1 wherein at least one of the plurality of electrodes is in close proximity to a site being stimulated and at least another one of the plurality of electrode is remote from the site being simulated.

3. The implantable digital stimulation system as recited in claim 1 further comprising a low impedance power generator with an internal reference.

4. The implantable digital stimulation system as recited in claim 3 wherein a distal electrode is related to a container of the power generator.

5. The implantable digital stimulation system as recited in claim 3 wherein a distal electrode is mounted on a container of the power generator.

6. The implantable digital stimulation system as recited in claim 1 wherein at least one of the plurality of electrodes is located at a distal coronary sinus, proximal coronary sinus, jugular vein, superior vena cava and inferior vena cava.

7. The implantable digital stimulation system as recited in claim 6 wherein during the stimulation treatment the implantable stimulator operates to:
pace the heart of the patient at a rate lower than the vagal stimulation rate, but faster than an intrinsic heart rate;
sense electrograms from one or more intravascular locations before and during producing stimulation signals;
compares morphology of the electrograms from the one or more intravascular locations before and during pacing the heart;
determining in response to morphology changes of the electrograms before and during pacing the heart, whether producing stimulation signals is stimulating a ventricle; and
continuing or stopping the stimulation treatment based on the morphology.

8. The implantable digital stimulation system as recited in claim 6 wherein the stimulation treatment comprises a high voltage vagal stimulation from the at least one intravascular location of the patient to slow ventricular rate to permit cardiac resynchronization therapy and prolong atrial-ventricular interval to allow greater filling time.

9. The vas recited in claim 6 wherein the stimulation treatment comprises a high voltage vagal stimulation from proximal part of the intravascular location of the patient to slow ventricular rate to permit cardiac resynchronization therapy.

10. The implantable digital stimulation system as recited in claim 1 further providing stimulation for ventricular defibrillation.

11. The implantable digital stimulation system as recited in claim 10 wherein during stimulation for ventricular defibrillation, the implantable stimulator operates to:
sense the heart rate of the patient prior to a stimulation;
stimulate of the vagal nerve for a predetermined time;
sense the heart rate during the stimulation of the vagal nerve; and
treat the patient for at least one of ventricular tachycardia, ventricular fibrillation, atrial fibrillation and supraventricular tachycardia, if the heart rate during the stimulation is not lower than the heart rate before the stimulation by a predetermined amount.

12. The implantable digital stimulation system as recited in claim 1 wherein the segmented waveforms are varied wither respect to shape, duration, and duty cycle.

13. The implantable digital stimulation system a recited in claim 1 wherein the sensing unit monitors an atrial rate of the patient.

14. The implantable digital stimulation system as recited in claim 1 wherein the sensing unit monitors a ventricular rate of the patient.

15. The implantable digital stimulation system as recited in claim 1 wherein the sensing unit monitors the atrial and ventricular rates of the patient.

16. The implantable digital stimulation system as recited in claim 1 wherein the atrial fibrillation treatment is performed by a high voltage pacing from the intravascular location using segmented waveforms of a predetermined rate, shape, duration and duty cycle.

17. An implantable apparatus for an electrical stimulation of a patient, the implantable apparatus comprising:
a low impedance power supply;
an implantable control unit for controlling the electrical stimulation by programmable selection of a treatment technique; and
at least two implantable stimulation electrodes that are programmably selectable by the control unit;
an implantable stimulation unit comprising:
(a) a stimulation signal generator producing stimulation waveforms that are programmably selectable by the control unit,
(b) an voltage intensifier that increases voltage of the stimulation waveforms and produces an output waveform that is applied to the at least two stimulation electrodes, and
(c) an sensing unit with an internal reference for monitoring a heart rate of the patient during treatment.

18. The implantable apparatus as recited in claim 17 wherein at least one of the stimulation electrodes is a proximate to the implantable stimulation unit and at least one of the stimulation electrodes is remote from the implantable stimulation unit.

19. The implantable apparatus as recited in claim 17 wherein the low impedance power supply is one of radio frequency based, piezoelectric device based, thermal energy source based, mechanical energy source based, and chemical energy source based.

20. The implantable apparatus as recited in claim 17 wherein the voltage intensifier is one of a flying capacitor-type voltage doubler, bipolar mode doubler, and a combination of capacitor type and bipolar mode voltage doubler.

21. The implantable apparatus as recited in claim 17 wherein the sensing unit is not connected to a common ground reference.

22. The implantable apparatus as recited in claim 17 wherein the stimulation unit varies duration of the output waveform to minimize losses.

23. The implantable apparatus as recited in claim 17 wherein the stimulation signal generator produces a compound multi-segmented waveform containing two or more waveform lobes.

24. An implantable apparatus for an electrical stimulation treatment of a patient, the implantable apparatus comprising:
a vagal nerve stimulation system comprising an implantable sensing unit with an internal reference to monitor a heart rate of the patient during vagal nerve stimulation and thereby produce an indication of a sensed heart rate; and
an implantable pacemaker to increase the heart rate of the patient if the sensed heart rate falls below a first predetermined threshold following the vagal stimulation.

25. The implantable apparatus as recited in claim 24 wherein the vagal nerve stimulation system further comprises:
a low impedance power supply;
an implantable control unit for controlling the electrical stimulation treatment;
a stimulation unit comprising implantable stimulation electrodes located at a vagal stimulation site, and an voltage intensifier; and
a waveform generator producing stimulation waveforms.
